(19) United States
(12) Patent Application Publication
(10) Pub. No.: US 2026/0202372 A1
(43) Pub. Date: Jul. 16, 2026

(54) BIOSENSOR WITH FLOATING GATE AND BACKSIDE CONTACT

(71) Applicant: INTERNATIONAL BUSINESS MACHINES CORPORATION, Armonk, NY (US)

(72) Inventors: Uzma Rana, Slingerlands, NY (US); Reinaldo Vega, Mahopac, NY (US); Takashi Ando, Eastchester, NY (US); Sufi Zafar, Briarcliff Manor, NY (US)

(21) Appl. No.: 19/021,360

(22) Filed: Jan. 15, 2025

(51) Int. Cl. G01N 27/30; G01N 27/22; G01N 33/543

(52) U.S. Cl. CPC G01N 27/301; G01N 27/22; G01N 33/54373

(57) ABSTRACT

Embodiments of present invention provide a biosensor structure. The biosensor structure includes a substrate having a frontside and a backside opposing the frontside; a sensing surface at the frontside of the substrate, the sensing surface being adapted to detect certain biomolecules in a test solution placed on the sensing surface; and a controlling surface at the backside of the substrate, the controlling surface providing access to a controlling terminal, and the controlling terminal being capacitively connected to the sensing surface. A method of forming the same is also provided.

### BACKGROUND

The present application relates to analytical devices from semiconductor structures. More particularly, it relates to a biosensor and method of manufacturing the biosensor.

Biosensors combine biological components with physicochemical detectors to detect analytes (i.e., chemical constituents that are of interest in an analytical procedure, such as ions and biomolecules). As such, biosensors play an important role in environmental applications and in the fields such as the food and healthcare industries. Some common examples of biosensors include, for example, blood glucose monitors and devices for detecting heavy metal ions and other contaminants in river water.

Field effect transistor (FET) based biosensors have demonstrated their ability for rapid and label-free detection of proteins, nucleotide sequences, and viruses at some ultra-low concentration levels, thereby having the potential to become a transformative diagnostic technology. Their nanoscale size gives the FET based biosensors their ultralow detection ability but, in the meantime, introduces the complexity of packaging and potential complication among controlling and sensing signals.

### SUMMARY

Embodiments of present invention provide a biosensor structure. The biosensor structure includes a substrate having a frontside and a backside opposing the frontside; a sensing surface at the frontside of the substrate, the sensing surface being adapted to detect certain biomolecules in a test solution placed on the sensing surface; and a controlling surface at the backside of the substrate, the controlling surface providing access to a controlling terminal, the controlling terminal being capacitively connected to the sensing surface.

According to one embodiment, the biosensor structure further includes a sensing transistor having a gate, a source region, and a drain region, where the sensing surface includes a sensing layer that is conductively connected to the gate of the sensing transistor.

According to another embodiment, the biosensor structure further includes a source contact and a drain contact that are respectively connected to the source region and the drain region of the sensing transistor, where the controlling surface provides accesses to the source contact and the drain contact at the backside of the substrate.

According to yet another embodiment, the biosensor structure further includes a dielectric layer formed at a bottom surface of the substrate, where the controlling surface provides accesses to the source contact, the drain contact, and the controlling terminal through one or more contact vias made in the dielectric layer.

In one embodiment, the controlling terminal is connected to the sensing surface through a controlling capacitor, the controlling capacitor being formed at the frontside of the substrate to include a first and a second conductive plate, the first and the second conductive plate being made of gold (Au) or polysilicon (poly-Si) and separated by a dielectric layer.

In another embodiment, the sensing surface includes a bio layer on top of a sensing layer, the sensing layer being conductively connected to a base of a bipolar-junction transistor (BTJ), and the bio layer being a monolayer of biotin proteins.

In yet another embodiment, the controlling terminal is conductively connected to a reference electrode, the reference electrode is made of silver (Ag) and capacitively connected to the sensing layer through a test solution.

According to one embodiment, the biosensor structure further includes a dummy transistor adjacent to the sensing transistor, where the controlling terminal is capacitively connected to the sensing layer through a capacitive structure formed by a gate and at least one of a source and a drain of the dummy transistor. In one aspect, the sensing transistor and the dummy transistor are either nanosheet transistors or fin-type FET transistors.

In one embodiment, the sensing layer is a metal line of a metal level in a back-end-of-line (BEOL) structure.

### BRIEF DESCRIPTION OF THE DRAWINGS

The present invention will be understood and appreciated more fully from the following detailed description of embodiments of present invention, taken in conjunction with accompanying drawings of which:

FIG. 1 is a demonstrative illustration of cross-sectional view of a biosensor according to a first embodiment of present invention;

FIGS. 2-6 are demonstrative illustrations of cross-sectional views of the biosensor illustrated in FIG. 1 during a process of manufacturing thereof according to embodiments of present invention;

FIG. 7 is a demonstrative illustration of cross-sectional view of a biosensor according to a second embodiment of present invention;

FIG. 8 is a demonstrative illustration of cross-sectional view of a biosensor according to a third embodiment of present invention;

FIG. 9 is a demonstrative illustration of cross-sectional view of a biosensor according to a fourth embodiment of present invention;

FIG. 10 is a demonstrative illustration of cross-sectional view of a biosensor according to a fifth embodiment of present invention; and

FIG. 11 is a demonstrative illustration of a flow-chart of a method of manufacturing a biosensor according to embodiments of present invention.

It will be appreciated that for simplicity and clarity purpose, elements shown in the drawings have not necessarily been drawn to scale. Further, and if applicable, in various functional block diagrams, two connected devices and/or elements may not necessarily be illustrated as being connected. In some other instances, grouping of certain elements in a functional block diagram may be solely for the purpose of description and may not necessarily imply that they are in a single physical entity, or they are embodied in a single physical entity.

### DETAILED DESCRIPTION

In the below detailed description and the accompanying drawings, it is to be understood that various layers, structures, and regions shown in the drawings are both demonstrative and schematic illustrations thereof that are not drawn to scale. In addition, for the ease of explanation, one or more layers, structures, and regions of a type commonly used to form semiconductor devices or structures may not be explicitly shown in a given illustration or drawing. This does not imply that any layers, structures, and regions not explicitly shown are omitted from the actual semiconductor structures. Furthermore, it is to be understood that the embodiments discussed herein are not limited to the particular materials, features, and processing steps shown and described herein. In particular, with respect to semiconductor processing steps, it is to be emphasized that the descriptions provided herein are not intended to encompass all of the processing steps that may be required to form a functional semiconductor integrated circuit device. Rather, certain processing steps that are commonly used in forming semiconductor devices, such as, for example, wet cleaning and annealing steps, are purposefully not described herein for economy of description.

It is to be understood that the terms “about” or “substantially” as used herein with regard to thicknesses, widths, percentages, ranges, etc., are meant to denote being close or approximate to, but not exactly. For example, the term “about” or “substantially” as used herein implies that a small margin of error may be present such as, by way of example only, 1% or less than the stated amount. Likewise, the terms “on”, “over”, or “on top of” that are used herein to describe a positional relationship between two layers or structures are intended to be broadly construed and should not be interpreted as precluding the presence of one or more intervening layers or structures.

Moreover, although various reference numerals may be used across different drawings, the same or similar reference numbers are used throughout the drawings to denote the same or similar features, elements, or structures, and thus detailed explanations of the same or similar features, elements, or structures may not be repeated for each of the drawings for economy of description. Labelling for the same or similar elements in some drawings may be omitted as well in order not to overcrowd the drawings.

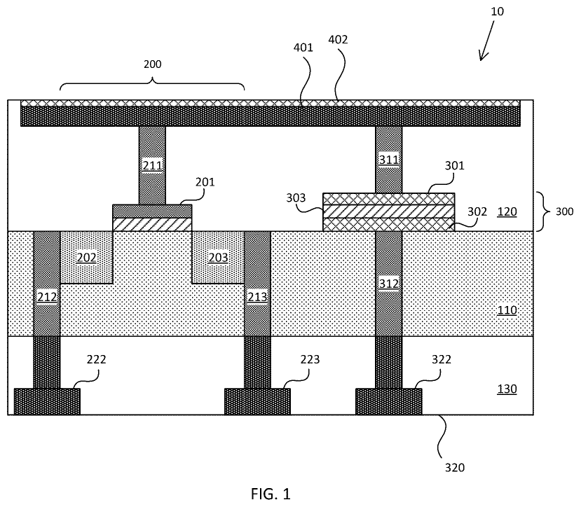

FIG. 1 is a demonstrative illustration of cross-sectional view of a biosensor according to a first embodiment of present invention. More particularly, the embodiment provides a biosensor structure 10. The biosensor structure 10 includes a semiconductor substrate 110 that has a frontside and a backside opposing the frontside. As is illustrated in FIG. 1, the frontside of the substrate 110 refers to a top side of the substrate 110 and the backside of the substrate 110 refers to a bottom side of the substrate 110.

At the frontside of the substrate 110, there may be formed a sensing transistor 200. The sensing transistor 200 may include a source region 202, a drain region 203, and a gate 201 that is formed over a channel region, via a gate dielectric layer, in the substrate 110 between the source region 202 and the drain region 203. At the frontside of the substrate 110, there also may be formed a controlling capacitor 300, or a capacitive structure, that includes a top conductive plate 301 and a bottom conductive plate 302. The top conductive plate 301 and the bottom conductive plate 302 may include or be made of gold (Au) or polysilicon (poly-Si), for example, and may be separated by a dielectric layer 303 such as silicon-oxide (SiOx), silicon-nitride (SiN), silicon-carbide (SiC), silicon-carbonitride (SiCN), or suitable dielectric materials.

Inside the substrate 110, there may be formed a source contact 212 in direct contact with the source region 202 and a drain contact 213 in direct contact with the drain region 203. Moreover, there is formed a controlling terminal 312, inside the substrate 110, that is in direct contact with the bottom conductive plate 302 of the controlling capacitor 300.

The biosensor structure 10 may also include a top dielectric layer 120 at the frontside of the substrate 110 and formed at a top surface thereof; and may also include a bottom dielectric layer 130 at the backside of the substrate 110 and formed at a bottom surface thereof. Embedded inside the top dielectric layer 120, there may be formed a gate contact 211 on top of and in direct contact with the gate 201 and a lead contact 311 in direct contact with the top conductive plate 301. The controlling capacitor 300 may be embedded in the dielectric layer 120. In one embodiment, the dielectric material of the top dielectric layer 120 may be the same as the dielectric layer 303 between the first and the second conductive plates 301 and 302.

At or near the top portion of the top dielectric layer 120, there is formed a sensing layer 401 that is formed in contact with both the gate contact 211 and the lead contact 311 of the controlling capacitor 300. The sensing layer 401 may be a layer of conductive material such as, for example, copper (Cu) and in one embodiment may be a metal line of a metal level of a back-end-of-line (BEOL). According to one embodiment a bio layer 402, such as a monolayer of biotin proteins, may be formed or coated on top of the sensing layer 401 to form a sensing surface. In other words, a sensing surface may be formed by the sensing layer 401 coated by the bio layer 402. The bio layer 402 helps bind target ions or biomolecules (to be detected) to the sensing layer 401 during sensing, detecting, or testing.

The biosensor structure 10 may be used in sensing or detecting certain biomolecules in a test solution. During the sensing or detecting process, the sensing surface may be adapted to receive or accept a test solution. The test solution may be placed on the sensing surface and made in contact with the sensing layer 401, through the bio layer 402, and may thus cause biochemical reaction in the sensing layer 401. This biochemical reaction in-turn affects, via the gate contact 211, a gate voltage at the gate 201. Any changes in gate voltage at the gate 201 may result in changes in current which may subsequently be detected between the source region 202 and the drain region 203. A control voltage applied to the controlling capacitor 300 at the controlling terminal 312 may also affect the gate voltage via the lead contact 311 and the sensing layer 401. The controlling terminal 312 is capacitively connected to the sensing layer 401 through the controlling capacitor 300.

According to one embodiment of present invention, the biosensor structure 10 may include a controlling surface 320, at a bottom surface of the bottom dielectric layer 130, that provides accesses to the sensing transistor 200 and the controlling terminal 312. More particularly, one or more contact vias such as a first contact via 322, a second contact via 222, and a third contact via 223 may be formed to be embedded in the bottom dielectric layer 130. The controlling surface 320 provides accesses to the source contact 212 and the drain contact 213 through the second contact via 222 and the third contact via 223 and provides access to the controlling terminal 312 through the first contact via 322.

By forming the controlling surface 320 at the backside of the substrate 110, more particularly at a bottom surface of the bottom dielectric layer 130, the biosensor structure 10 is enabled to have more sensing area at the top of the sensing layer 401 and avoid complication in, for example, co-packaging the sensing surface with the controlling surface 320, both at the frontside of the substrate 110, to provide access to the controlling terminal 312. By separating the sensing surface from the controlling surface to the frontside and backside of the substrate 110, additional complication may be removed or avoided as well such as, for example, operational interference between the sensing layer 401 and the controlling surface 320; decreasing in sensitivity due to reduced sensing area of the sensing layer 401; and compactness or size of the overall biosensor structure 10.

As being discussed above, accesses to the sensing transistor 200 including the source region 202 and the drain region 203 may be provided by the controlling surface 320 at the backside of the substrate 110. This further reduces the overall demand for real estate at the frontside of the substrate 110. For example, access to the source contact 212 may be provided through the second contact via 222, and access to the drain contact 213 may be provided through the third contact via 223. In other words, the first, second, and third contact vias 322, 222, and 223, embedded in the bottom dielectric layer 130, provide accesses to the source region 202 and the drain region 203, via the source contact 212 and the drain contact 213, and access to the controlling terminal 312.

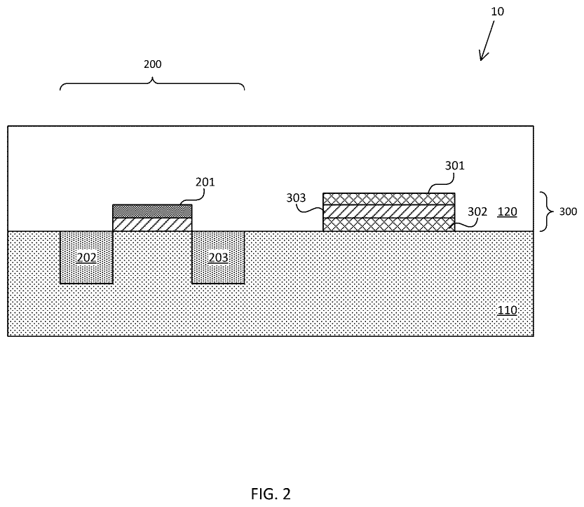

FIGS. 2-6 are demonstrative illustrations of cross-sectional views of the biosensor structure 10 illustrated in FIG. 1 during a process of manufacturing thereof according to embodiments of present invention. More particularly, as is illustrated in FIG. 2, in forming the biosensor structure 10, embodiments of present invention provide receiving or providing a semiconductor substrate 110 and forming a top dielectric layer 120 on a top surface of the substrate 110 at a frontside thereof. At the frontside of the substrate 110 and embedded in the top dielectric layer 120, embodiments of present invention provide forming a sensing transistor 200 that includes a source region 202, a drain region 203, and a gate 201 on top of a channel region between the source region 202 and the drain region 203 in the substrate 110. Embodiments of present invention may further provide forming a controlling capacitor 300, embedded in the top dielectric layer 120 at the frontside of the substrate 110. The controlling capacitor 300 may be formed to include a top (or a first) and a bottom (or a second) conductive plate 301 and 302 with a dielectric layer 303 in between the top and the bottom conductive plate 301 and 302. The top and the bottom conductive plate 301 and 302 may be metal, such as gold (Au), or polysilicon (poly-Si), the top dielectric layer 120 and the dielectric layer 303 between the top an the bottom conductive plate 301 and 302 may be, for example, SiOx, SiN, SiCN, or other suitable material and in one embodiment may be a same dielectric material.

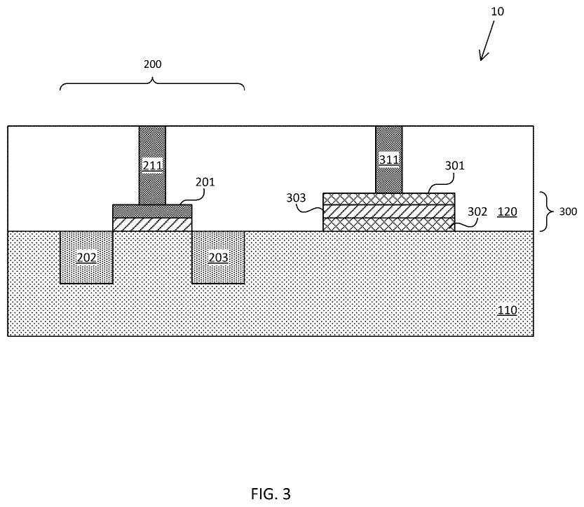

FIG. 3 is a demonstrative illustration of cross-sectional view of a biosensor structure during a process of manufacturing thereof, following the step illustrated in FIG. 2, according to embodiments of present invention. More particularly, embodiments of present invention further provide forming a gate contact 211 and a lead contact 311 inside the top dielectric layer 120 that, respectively, contact the gate 201 of the sensing transistor 200 and the top conductive plate 301 of the controlling capacitor 300. The formation of the gate contact 211 and the lead contact 311 may be made through, for example, a lithographic patterning and etching process by first creating openings inside the top dielectric layer 120, and subsequently depositing conductive material such as, copper (Cu), cobalt (Co), or aluminum (Al) or other suitable material inside the openings to form the gate contact 211 and the lead contact 311. The deposition of the conductive material may be made through, for example, a chemical-vapor-deposition (CVD) process, a physical-vapor-deposition (PVD) process, and/or an atomic-layer-deposition (ALD) process.

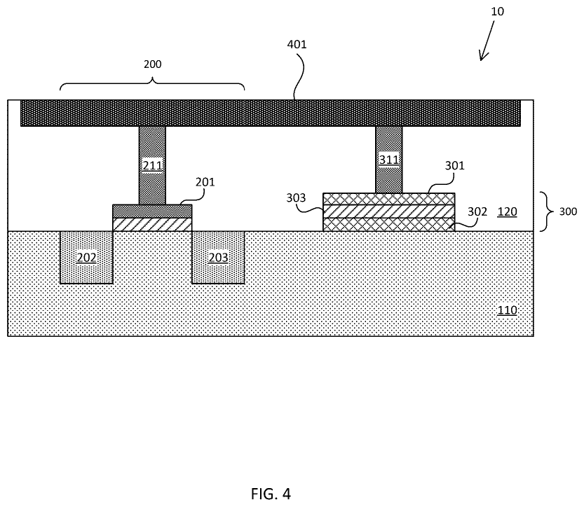

FIG. 4 is a demonstrative illustration of cross-sectional view of a biosensor structure during a process of manufacturing thereof, following the step illustrated in FIG. 3, according to embodiments of present invention. More particularly, embodiments of present invention further provide forming a sensing layer 401 on top of and in contact with the gate contact 211 and the lead contact 311. The sensing layer 401 may be a layer of conductive material such as, for example, Cu, Co, Al and maybe formed on top of or embedded in the top dielectric layer 120. Additionally, a bio layer (now shown) may be formed on top of the sensing layer 401. The bio layer may be, in one embodiment and for example, a layer of biotin proteins may, together with the sensing layer 401, form a sensing surface.

During sensing, detecting, or testing, the sensing surface may be adapted to receive or accept a test solution. The test solution may be placed on the sensing surface and may cause biochemical reactions in the sensing layer 401. The biochemical reactions in-turn may affect, via the gate contact 211, a gate voltage at the gate 201, resulting in changes in current that may flow between the source region 202 and the drain region 203 and may be detected. With the lead contact 311 being in contact with the sensing layer 401, a control voltage that is capacitively applied to the lead contact 311 of the controlling capacitor 300 as being described below in more details, may be used to influence or control the operation of the biosensor structure 10 and in particular the sensing transistor 200.

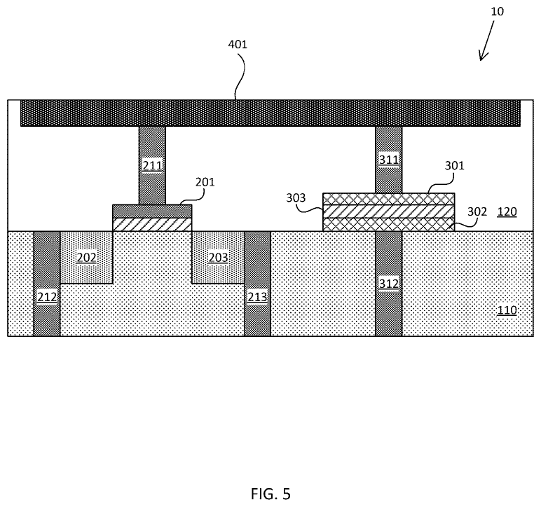

FIG. 5 is a demonstrative illustration of cross-sectional view of a biosensor structure during a process of manufacturing thereof, following the step illustrated in FIG. 4, according to embodiments of present invention. More particularly, embodiments of present invention further provide flipping the substrate 110 upside-down and forming a source contact 212 and a drain contact 213 of the sensing transistor 200, and a controlling terminal 312 of the controlling capacitor 300 from the backside of the substrate 110. Here, in order to avoid causing confusion and for the sake of consistency in illustration, the biosensor structure 10 including the substrate 110 will continue to be illustrated in an upside-up manner, but the manufacturing process described hereinafter should be understood as being performed from the backside or bottom side of the substrate 110.

For example, one or more openings such as through-silicon-via (TSV) openings may be created or etched in and through the substrate 110. The one or more openings may be created through a lithographic patterning and etching process. The one or more openings may be created to expose at least a portion of the source region 202, the drain region 203, and the bottom conductive plate 302 of the controlling capacitor 300. Subsequently, conductive materials such as Cu, Co, Al, or other suitable conductive materials may be deposited to fill the one or more openings thereby forming the source contact 212, the drain contact 213, and the controlling terminal 312. The deposition of the conductive material may be made through, for example, a CVD process, a PVD process, an ALD process, or any other currently existing or future developed suitable deposition process. After the deposition, a chemical-mechanical-polishing (CMP) process may be applied to polish off any excess of the conductive material that may be left on top of the substrate 110 (i.e., at the bottom surface of the substrate 110 as being illustrated in FIG. 5), thereby resulting the source contact 212, drain contact 213, and the controlling terminal 312 to be coplanar with the substrate 110. In one embodiment, the substrate 110 may be further polished and/or grinded to reduce an overall thickness.

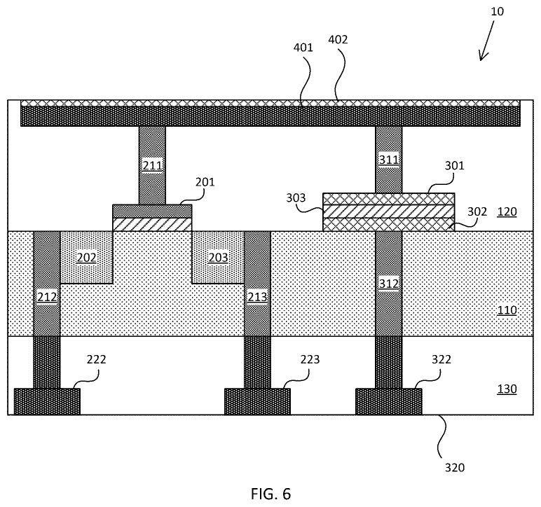

FIG. 6 is a demonstrative illustration of cross-sectional view of a biosensor structure during a process of manufacturing thereof, following the step illustrated in FIG. 5, according to embodiments of present invention. More particularly, embodiments of present invention further provide forming a controlling surface 320 that provides backside access to the controlling terminal 312, and possibly backside accesses to the source contact 212 and the drain contact 213 of the sensing transistor 200.

For example, in one embodiment, one or more via openings may be created in a separate dielectric layer. Next, conductive material may be deposited to fill the one or more via openings thereby forming a first contact via 322, a second contact via 222, and a third contact via 223. After forming the first, second, and third contact vias 322, 222, and 223, this dielectric layer may be bonded, for example through a thermal bonding process, onto the substrate 110 to become a bottom dielectric layer 130 while ensuring that the first contact via 322, the second contact via 222, and the third contact via 223 are respectively and substantially aligned with the controlling terminal 312, the source contact 212, and the drain contact 213.

Further for example, in another embodiment, a bottom dielectric layer 130 may first be formed, through a deposition process, at a bottom surface of the substrate 110. Next, one or more via openings may be created, in the bottom dielectric layer 130, that are respectively aligned with, thereby expose, the controlling terminal 312, the source contact 212, and the drain contact 213. Conductive material may subsequently be deposited in these via openings to form contact vias such that a first contact via 322 is in contact with the controlling terminal 312, a second contact via 222 is in contact with the source contact 212, and a third contact via 223 is in contact with the drain contact 213.

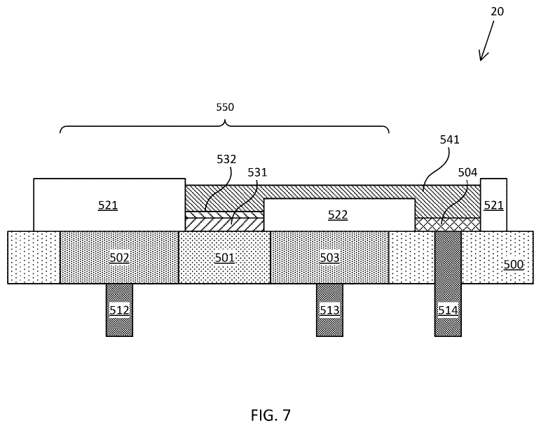

FIG. 7 is a demonstrative illustration of cross-sectional view of a biosensor structure according to a second embodiment of present invention. More particularly, FIG. 7 illustrates a biosensor structure 20 that applies a bipolar junction transistor (BTJ) as a sensing transistor. For example, the biosensor structure 20 may include a BTJ 550 formed in a substrate 500. The BTJ 550 includes an emitter 502, a collector 503 and a base 501 in-between the emitter 502 and the collector 503. Contacts 512 and 513 may be formed in contact with the emitter 502 and the collector 503 respectively. Dielectric material may be formed on top of the emitter 502 and the collector 503 to expose only area of the base 501. For example, a first oxide layer 521 may be formed that fully covers the emitter 502 and a second oxide layer 522 may be formed that fully covers the collector 503. A sensing layer 531 may be formed on top of the base 501 and insulated from the emitter 502 by the first oxide layer 521 and insulated from the collector 503 by the second oxide layer 522. The sensing layer 531 may be a layer of conductive material such as, for example, titanium-nitride (TiN), gold (Au), to name a few. A bio layer 532 may be formed on top of the sensing layer 531. The bio layer 532 may be, for example, a monolayer of biotin proteins and helps bind target ions or biomolecules to the sensing layer 531 during their sensing, detecting, or testing.

The biosensor structure 20 may further include a reference electrode 504 formed on top of the substrate 500. During sensing, detecting, or testing, the sensing surface of the bio layer 532 and the sensing layer 531 may be adapted to receive or accept a test solution 541, and the reference electrode 504 may be capacitively connected with the sensing surface through the test solution 541. The test solution 541 placed on top of the sensing layer 531, the bio layer 532, and the reference electrode 504 may be enclosed by the first oxide layer 521, which has a height higher than the second oxide layer 522. The reference electrode 504 may be made of, for example, silver (Ag), silver-chlorine (AgCl), Ag coated by AgCl, or other suitable materials.

According to one embodiment, a controlling terminal 514 may be formed from a backside of the substrate 500 to be in contact with the reference electrode 504. By forming the controlling terminal 514 from the backside of the substrate 500, the biosensor structure 20 may have more spaces at the frontside of the substrate 500. The spaces may be used for forming a larger sensing layer 531, resulting in more sensing areas for increased sensitivity. Forming the controlling terminal 514 at the backside of the substrate 500 may also bring other benefits, such as those being discussed above with regard to the biosensor structure 10.

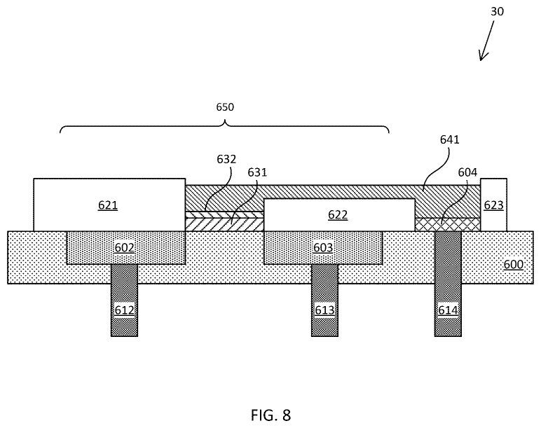

FIG. 8 is a demonstrative illustration of cross-sectional view of a biosensor structure according to a third embodiment of present invention. More particular, FIG. 8 illustrates a biosensor structure 30 that applies a field-effect-transistor (FET) such as a planar FET as a sensing transistor. For example, the biosensor structure 30 may include a FET 650 formed in a substrate 600. The FET 650 includes a source region 602 (or drain region), a drain region 603 (or source region), and a channel region between the source region 602 and the drain region 603. Contacts 612 and 613 may be formed in contact with the source region 602 and the drain region 603 respectively. A gate 631 may be formed, via a gate dielectric layer, on top of and covering the channel region in the substrate 600 between the source region 602 and the drain region 603. Additionally, a bio layer 632 may be formed directly on top of the gate 631. The bio layer 632 may be formed to bind target biomolecules to the gate 631. For example, the bio layer 632 may be a monolayer of biotin proteins being attached to the gate 631 for detecting target protein such as, for example, streptavidin. Together, the bio layer 632 and the gate 631 form a sensing surface, with the gate 631 serving as a sensing layer.

Like the biosensor structure 20, dielectric material may be formed on top of the source region 602 and the drain region 603 leaving only the bio layer 632 on top of the gate 631 being exposed. For example, a first oxide layer 621 may be formed that fully covers the source region 602, a second oxide layer 622 may be formed that fully covers the drain region 603, and a third oxide layer 623 may be formed directly on top of the substrate 600 and, as is illustrated in FIG. 8, to the right of a reference electrode 604 as being described below in more details. The first and the third oxide layers 621 and 623 may have a height that is higher than a height of the second oxide layer 622, thereby may be able to hold and enclose a test solution 641 during a sensing or testing process.

The biosensor structure 30 may further include the reference electrode 604 formed on top of the substrate 600. During a sensing, detecting, or testing process, the sensing surface of the bio layer 632 and the gate 631 may be adapted to receive or accept the test solution 641, and the reference electrode 604 may be capacitively connected to the gate 631 via the test solution 641. Because of the height difference, the test solution 641 may be enclosed by the first oxide layer 621 and the third oxide layer 623, while the test solution 641 may be placed on top of the gate 631 via the bio layer 632, on top of the reference electrode 604, and on top of the second oxide layer 622. The reference electrode 604 may be made of, for example, Ag, AgCl, Ag coated by AgCl, or other suitable materials.

According to one embodiment, a controlling terminal 614 may be formed from a backside of the substrate 600 to be in contact with the reference electrode 604. By forming the controlling terminal 614 from the backside of the substrate 600, the biosensor structure 30 may have more spaces at the frontside of the substrate 600. The spaces may be used for forming a larger bio layer 632, resulting in more sensing areas for increased sensitivity of the biosensor structure 30, and other benefits as described above with regard to the biosensor structure 10, and the biosensor structure 20.

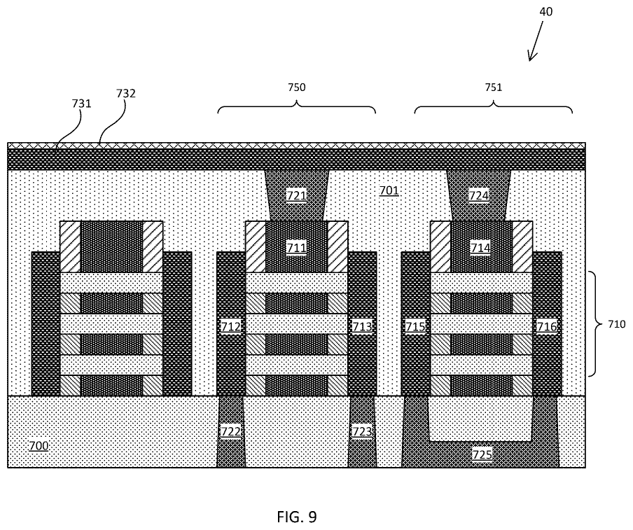

FIG. 9 is a demonstrative illustration of cross-sectional view of a biosensor structure according to a fourth embodiment of present invention. More particular, FIG. 9 illustrates a biosensor structure 40 that applies a FET such as a nanosheet transistor (NSFET) as a sensing transistor. For example, the biosensor structure 40 may include a sensing transistor of a first NSFET 750 formed above a substrate 700 and embedded in a dielectric layer 701. The first NSFET 750 includes a set of nanosheets 710 wrapped around by a metal gate 711, and a source region 712 (or drain region) and a drain region 713 (or source region) at two opposing ends of the set of nanosheets 710. A gate contact 721 is formed in contact with the metal gate 711, and a sensing surface is formed on top of the dielectric layer 701. The sensing surface may include a bio layer 732 on top of a sensing layer 731, and the sensing layer 731 is in contact with the metal gate 711 through the gate contact 721.

According to one embodiment, a second NSFET 751 may be used as a controlling circuit for controlling the sensing transistor of the first NSFET 750. The second NSFET 751 may be known as a dummy NSFET and may be adjacent to the first NSFET 750. The second NSFET 751 may include a source region 715, a drain region 716, and a metal gate 714. This second NSFET 751 may have a capacitive structure with a lead contact 724 in contact with the metal gate 714. A controlling terminal 725, to be described later, may be in contact with at least one of the source region 715 and the drain region 716. The lead contact 724 is formed in the dielectric layer 701 and conductively connected to the sensing layer 731, while the controlling terminal 725 is capacitively connected to the sensing layer 731 via the capacitive structure of the second NSFET 751 between the metal gate 714 and the at least one of the source region 715 and the drain region 716 of the second NSFET 751.

According to one embodiment, the controlling terminal 725 may be formed from a backside of the substrate 700 to be in contact with at least one of the source region 715 and the drain region 716 of the second NSFET 751. By forming the controlling terminal 725 at the backside of the substrate 700, the biosensor structure 40 may have more spaces at the frontside of the substrate 700 for forming the sensing layer 731. In addition, a source contact 722 and a drain contact 723 may be formed from the backside of the substrate 700 to conductively contact the source region 712 and the drain region 713 of the first NSFET 750 respectively. Forming the source contact 722 and the drain contact 723 from the backside of the substrate 700 further helps improving the availability of real estate at the frontside of the substrate 700 for forming a larger sensing area of the sensing layer 731 and avoids much of the complications being described above with regard to the biosensor structures 10, 20, and 30.

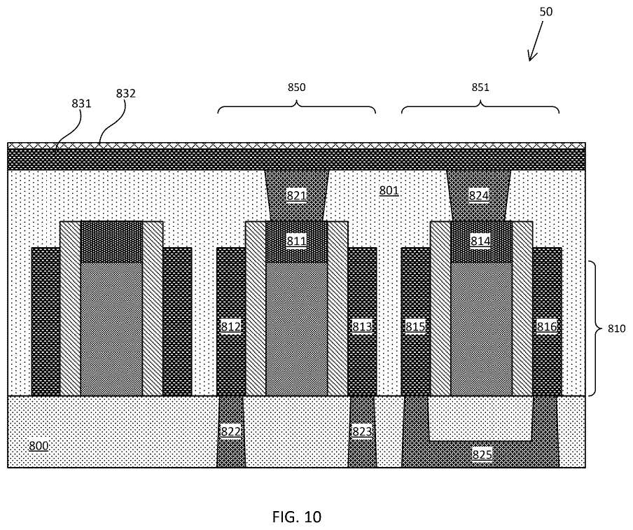

FIG. 10 is a demonstrative illustration of cross-sectional view of a biosensor structure according to a fifth embodiment of present invention. More particular, FIG. 10 illustrates a biosensor structure 50 that applies a FET such as a fin-type FET (FinFET) as a sensing transistor. For example, the biosensor structure 50 may include a sensing transistor of a first FinFET 850 formed above a substrate 800 and embedded in a dielectric layer 801. The first FinFET 850 includes a fin-shaped channel region 810 and a metal gate 811 saddled on top of thereby surrounding the fin-shaped channel region 810. The first FinFET 850 further includes a source region 812 (or drain region) and a drain region 813 (or source region) at two opposing ends of the fin-shaped channel region 810. A gate contact 821 is formed in contact with the metal gate 811 and sensing surface is formed on top of the dielectric layer 801. The sensing surface may include a bio layer 832 on top of a sensing layer 831, and the sensing layer 831 is in contact with the metal gate 811 through the gate contact 821.

According to one embodiment, a second FinFET 851 may be used as a controlling circuit for controlling the sensing transistor of the first FinFET 850. The second FinFET 851 may be known as a dummy FinFET and may be adjacent to the first FinFET 850. The second FinFET 851 may include a source region 815, a drain region 816, and a metal gate 814. This second FinFET 851 may have a capacitive structure with a lead contact 824 in contact with the metal gate 814. A controlling terminal 825, to be described later, may be in contact with at least one of the source region 815 and the drain region 816. The lead contact 824 is formed in the dielectric layer 801 and conductively connected to the sensing layer 831, while the controlling terminal 825 is capacitively connected to the sensing layer 831 via the capacitive structure of the second FinFET 851 between the metal gate 814 and the at least one of the source region 815 and the drain region 816 of the second FinFET 851.

According to one embodiment, the controlling terminal 825 may be formed from a backside of the substrate 800 to be in contact with at least one of the source region 815 and the drain region 816 of the second FinFET 851. By forming the controlling terminal 825 at the backside of the substrate 800, the biosensor structure 50 may have more spaces at the frontside of the substrate 800 for forming the sensing layer 831. In addition, a source contact 822 and a drain contact 823 may be formed from the backside of the substrate 800 to conductively contact the source region 812 and the drain region 813 of the first FinFET 850 respectively. Forming the source contact 822 and the drain contact 823 from the backside of the substrate 800 further helps improving the availability of real estate at the frontside of the substrate 800 for forming a larger sensing area of the sensing layer 831 and avoids much of the complications being described above with regard to the biosensor structures 10, 20, 30, and 40.

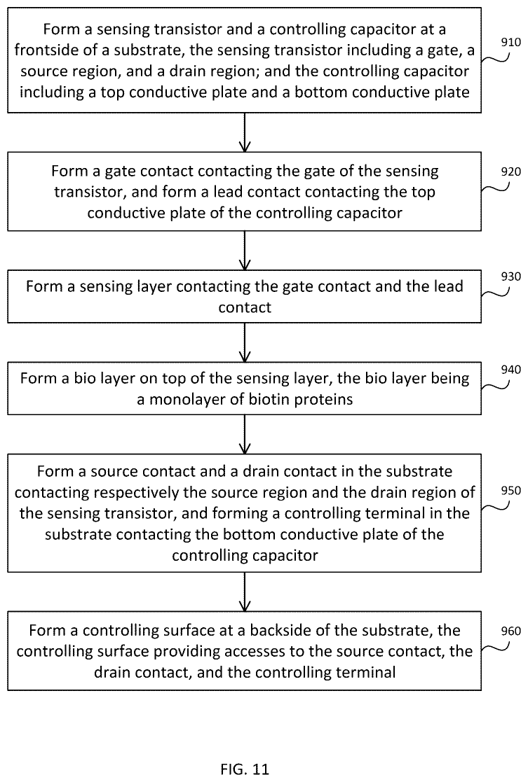

FIG. 11 is a demonstrative illustration of a flow-chart of a method of manufacturing a biosensor according to embodiments of present invention. The method includes a step (910) of forming a sensing transistor and a controlling capacitor at a frontside of a substrate, the sensing transistor including a gate, a source region, and a drain region; and the controlling capacitor including a top conductive plate and a bottom conductive plate; a step (920) of forming a gate contact contacting the gate of the sensing transistor, and form a lead contact contacting the top conductive plate of the controlling capacitor; a step (930) of forming a sensing layer contacting the gate contact and the lead contact; a step (940) of forming a bio layer on top of the sensing layer, the bio layer being a monolayer of biotin proteins; a step (950) of forming a source contact and a drain contact in the substrate contacting respectively the source region and the drain region of the sensing transistor, and forming a controlling terminal in the substrate contacting the bottom conductive plate of the controlling capacitor; and a step (960) of forming a controlling surface at a backside of the substrate, the controlling surface providing accesses to the source contact, the drain contact, and the controlling terminal.

It is to be understood that the exemplary methods discussed herein may be readily incorporated with other semiconductor processing flows, semiconductor devices, and integrated circuits with various analog and digital circuitry or mixed-signal circuitry. In particular, integrated circuit dies can be fabricated with various devices such as field-effect transistors, bipolar transistors, metal-oxide-semiconductor transistors, diodes, capacitors, inductors, etc. An integrated circuit in accordance with the present invention can be employed in applications, hardware, and/or electronic systems. Suitable hardware and systems for implementing the invention may include, but are not limited to, personal computers, communication networks, electronic commerce systems, portable communications devices (e.g., cell phones), solid-state media storage devices, functional circuitry, etc. Systems and hardware incorporating such integrated circuits are considered part of the embodiments described herein. Given the teachings of the invention provided herein, one of ordinary skill in the art will be able to contemplate other implementations and applications of the techniques of the invention.

Accordingly, at least portions of one or more of the semiconductor structures described herein may be implemented in integrated circuits. The resulting integrated circuit chips may be distributed by the fabricator in raw wafer form (that is, as a single wafer that has multiple unpackaged chips), as a bare die, or in a packaged form. In the latter case the chip may be mounted in a single chip package (such as a plastic carrier, with leads that are affixed to a motherboard or other high-level carrier) or in a multichip package (such as a ceramic carrier that has surface interconnections and/or buried interconnections). In any case the chip may then be integrated with other chips, discrete circuit elements, and/or other signal processing devices as part of either an intermediate product, such as a motherboard, or an end product. The end product may be any product that includes integrated circuit chips, ranging from toys and other low-end applications to advanced computer products having a display, a keyboard or other input device, and a central processor.

The descriptions of various embodiments of present invention have been presented for the purposes of illustration and they are not intended to be exhaustive and present invention are not limited to the embodiments disclosed. The terminology used herein was chosen to best explain the principles of the embodiments, practical application or technical improvement over technologies found in the marketplace, and to enable others of ordinary skill in the art to understand the embodiments disclosed herein. Many modifications, substitutions, changes, and equivalents will now occur to those of ordinary skill in the art. Such changes, modification, and/or alternative embodiments may be made without departing from the spirit of present invention and are hereby all contemplated and considered within the scope of present invention. It is, therefore, to be understood that the appended claims are intended to cover all such modifications and changes as fall within the spirit of the invention.

What is claimed is:

1. A biosensor structure comprising:
a substrate having a frontside and a backside opposite the frontside;
a sensing surface at the frontside of the substrate, the sensing surface being adapted to detect certain biomolecules in a test solution placed on the sensing surface; and
a controlling surface at the backside of the substrate, the controlling surface providing access to a controlling terminal, the controlling terminal being capacitively connected to the sensing surface.

2. The biosensor structure of claim 1, further comprising a sensing transistor having a gate, a source region, and a drain region, wherein the sensing surface includes a sensing layer that is conductively connected to the gate of the sensing transistor.

3. The biosensor structure of claim 2, further comprising a source contact and a drain contact that are respectively connected to the source region and the drain region of the sensing transistor, wherein the controlling surface provides accesses to the source contact and the drain contact at the backside of the substrate.

4. The biosensor structure of claim 3, further comprising a dielectric layer formed at a bottom surface of the substrate, wherein the controlling surface provides accesses to the source contact, the drain contact, and the controlling terminal through one or more contact vias made in the dielectric layer.

5. The biosensor structure of claim 1, wherein the controlling terminal is connected to the sensing surface through a controlling capacitor, the controlling capacitor being formed at the frontside of the substrate to include a first and a second conductive plate, the first and the second conductive plate being made of gold (Au) or polysilicon (poly-Si) and separated by a dielectric layer.

6. The biosensor structure of claim 1, wherein the sensing surface includes a bio layer on top of a sensing layer, the sensing layer being conductively connected to a base of a bipolar junction transistor (BTJ), and the bio layer being a monolayer of biotin proteins.

7. The biosensor structure of claim 6, wherein the controlling terminal is conductively connected to a reference electrode, the reference electrode is made of silver (Ag), coated by AgCl, and capacitively connected to the sensing layer through a test solution.

8. The biosensor structure of claim 2, further comprising a dummy transistor adjacent to the sensing transistor, wherein the controlling terminal is capacitively connected to the sensing layer through a capacitive structure formed by a gate and at least one of a source and a drain of the dummy transistor.

9. The biosensor structure of claim 8, wherein the sensing transistor and the dummy transistor are either nanosheet transistors or FinFET transistors.

10. The biosensor structure of claim 8, wherein the sensing layer is a metal line of a metal level in a back-end-of-line (BEOL) structure.

11. A method of forming a biosensor structure, the method comprises:
forming a sensing transistor and a controlling capacitor at a frontside of a substrate, the sensing transistor including a gate, a source region, and a drain region; and the controlling capacitor including a top conductive plate and a bottom conductive plate;
forming a gate contact contacting the gate of the sensing transistor, and forming a lead contact contacting the top conductive plate of the controlling capacitor;
forming a sensing layer contacting the gate contact and the lead contact;
forming a source contact and a drain contact in the substrate contacting respectively the source region and the drain region of the sensing transistor, and forming a controlling terminal in the substrate contacting the bottom conductive plate of the controlling capacitor; and
forming a controlling surface at a backside of the substrate, the controlling surface providing accesses to the source contact, the drain contact, and the controlling terminal.

12. The method of claim 11, further comprising forming a bio layer on top of the sensing layer, the bio layer being a monolayer of biotin proteins.

13. The method of claim 12, wherein forming the controlling surface comprises forming a dielectric layer at a bottom surface of the substrate and forming one or more vias in the dielectric layer contacting the source contact, the drain contact, and the controlling terminal.

14. A biosensor structure comprising:
a substrate having a frontside and a backside opposing the frontside;
a sensing transistor at the frontside of the substrate;
a sensing surface above the sensing transistor, the sensing surface including a sensing layer conductively connected to a gate of the sensing transistor; and
a controlling surface at the backside of the substrate, the controlling surface providing access to a controlling terminal capacitively connected to the sensing surface, and accesses to a source region and a drain region of the sensing transistor.

15. The biosensor structure of claim 14, further comprising a source contact and a drain contact in the substrate conductively connected, respectively, to the source region and the drain region of the sensing transistor.

16. The biosensor structure of claim 15, further comprising a dielectric layer formed at a bottom surface of the substrate, wherein the controlling surface is at a bottom surface of a dielectric layer, and provides accesses to the source contact, the drain contact, and the controlling terminal through one or more contact vias made in the dielectric layer.

17. The biosensor structure of claim 14, wherein the controlling terminal is connected to the sensing surface through a capacitive structure, the capacitive structure being formed at the frontside of the substrate to include at least one conductive plate.

18. The biosensor structure of claim 17, wherein the sensing surface includes bio layer on top of a sensing layer with the sensing layer being conductively connected to a base of a bipolar junction transistor and the bio layer being a monolayer of biotin proteins, and wherein the at least one conductive plate is a reference electrode, the reference electrode comprising silver.

19. The biosensor structure of claim 14, further comprising a dummy transistor adjacent to the sensing transistor, wherein the controlling terminal is capacitively connected to the sensing surface through a capacitive structure formed between a gate and at least one of a source and a drain of the dummy transistor.

20. The biosensor structure of claim 19, wherein the sensing transistor and the dummy transistor are either nanosheet transistors or FinFET transistors.
